(19) United States
(12) Patent Application Publication
(10) Pub. No.: US 2010/0305391 A1
(43) Pub. Date: Dec. 2, 2010

(54) MAGNOTHERAPY

(76) Inventor: Derek Raymond Price, Wiltshire (GB)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 12/239,032

(22) Filed: Sep. 26, 2008

Related U.S. Application Data
(60) Provisional application No. 60/975,359, filed on Sep. 26, 2007.

(51) Int. Cl. A61N 2/08

(52) U.S. Cl. 600/9

(57) ABSTRACT

The present invention relates to the use of magnets in the treatment of medical conditions wherein the magnotherapy product comprises a magnet having positive and negative poles, the magnet having a metallic element which distorts the magnetic field distribution around the magnet so as to attenuate the magnetic field in the vicinity of the positive pole of the magnet. The invention also relates to methods of treatment using those magnets.

### FIELD OF INVENTION

The present invention relates to the use of magnets in the treatment of medical conditions wherein the magnotherapy product comprises a magnet having positive and negative poles, the magnet having a metallic element which distorts the magnetic field distribution around the magnet so as to attenuate the magnetic field in the vicinity of the positive pole of the magnet. The invention also relates to methods of treatment using those magnets.

### BACKGROUND

Magnotherapy describes the practice of placing magnets adjacent an animal or human body in order to cure a disease, alleviate symptoms or to enhance performance of the animal or human. The mechanism by which magnotherapy works has to date not been understood or confirmed by the scientific community. A discussion of magnotherapy can be found in the book “Magnetic Health, Modern Day Healing with Magnets” by D R Price (ISBN0953679705).

Restless Legs Syndrome (RLS) is a term coined by Professor Karl-Axel Ekbom in 1944 and is therefore also known as “Ekbom's disease”. RLS is a neurological disorder in which a sufferer experiences unpleasant sensations in the legs and an uncontrollable urge to move when at rest to try to relieve these sensations and urges. RLS sensations are often described by people as burning, creeping, tugging, or like insects crawling inside the legs, and a wide variety of descriptions are included in diagnostic criteria. Often called paresthesias (abnormal sensations) or dysesthesias (unpleasant abnormal sensations), the sensations range in severity from uncomfortable to irritating to painful. Lying down and trying to relax activates the symptoms or makes them worse. Most people with RLS have difficulty falling asleep and staying asleep and may therefore be exhausted with daytime fatigue and sleepiness.

The prevalence of RLS in the general UK population is between 3% and 9% (Trenkwalder C et al, 2005, Lancet Neurol. 4(8): 465-75). This represents an estimated 2 to 5.5 million sufferers of RLS in the UK. There are an estimated 12 million sufferers in the USA (National Institute of Neurological Disorders Website, www.ninds.nih.gov/disorders/restless_legs/). RLS may be under-diagnosed and, in some cases, misdiagnosed. Some people with RLS will not seek medical attention, believing that they will not be taken seriously, that their symptoms are too mild, or that their condition is not treatable. Some physicians wrongly attribute the symptoms to nervousness, insomnia, stress, arthritis, muscle cramps, or aging.

In most cases, the cause of RLS is unknown (referred to as “idiopathic”). A family history of the condition is seen in approximately 50 percent of such cases, suggesting a genetic form of the disorder. People with familial RLS tend to be younger when symptoms start and have a slower progression of the condition. Despite efforts to establish standard criteria, the clinical diagnosis of RLS is difficult to make.

A diagnosis of RLS is normally given when the following four main symptoms are present:
Tingling, numbness,
Pain and/or a deep ache in the limbs,
Symptoms worsen with inactivity, symptoms worsen at night,
Sometimes causing sleep problems, and restlessness is eased when the limb is moved.

RLS is difficult to treat and the level of relief and consistency of effect with existing treatments is generally very variable. Furthermore, no one drug is effective for everyone with RLS. What may be helpful to one individual may actually worsen symptoms for another. In addition, medications taken regularly may lose their effect, making it necessary to change medications periodically. Mirapexin has been approved for use by the European Medicines Agency to treat moderate or severe cases. There are currently no other medications licensed for the treatment of RLS in the United Kingdom.

Although current primary and secondary prophylaxis have made substantial contributions to the management of cardiovascular disease, to date both medical and surgical approaches have failed to adequately address either the ubiquity or the mortality of the disease. These management problems continue to stimulate interest in the development of new approaches to therapy, including those of a complementary and alternative nature.

Cardiovascular disease is a significant cause of disability and death. It may present clinically as myocardial ischaemia, stroke, and peripheral vascular diseases. Mortality is principally a consequence of ischaemic heart disease.

Peripheral vascular disease (PVD) is a result of stenotic occlusion of large peripheral arteries consequent upon changes in the vascular wall, which reduces blood flow to the limb. The most common of the serious peripheral vascular diseases are the occlusive arterial diseases (PAOD).

In the Western world, mortality is generally a consequence of coronary artery and cerebrovascular disease, whereas PAOD is largely a morbid condition causing chronic disability and pain with a consequent reduction in the quality of life. However, as the same risk factors exist for both coronary heart disease (CHD) and PVD, they frequently coexist. Moreover, it has been recognised that mortality of patients with concomitant PVD and CHD is substantially greater than that in patients with PVD free from coronary ischaemic manifestations.

Atherosclerosis is a dynamic process that involves interactions between the vascular wall and the blood and also the activation of a number of biological systems. The essential components of the disease pathogenesis are repeated damage to vascular endothelial cells, coagulation, the deposition, migration and esterification of lipids within the vascular wall and the proliferation of vascular smooth muscle cells. Normally, vascular endothelial cells exist as a continuous, anti-thrombotic and anti-inflammatory sheet lining the blood vessels, Apoptosis (programmed cell death) and regeneration are physiological processes that contribute to vessel haemostasis by eliminating damaged cells from the vessel wall. However, increased turnover mediated through accelerated apoptosis appears to lead to dysfunction and promote atherosclerosis. An arterial atherosclerotic plaque is known as ‘atheroma’. Atheroma lead to alterations in arterial elasticity, luminal narrowing, vasospasm and a reduction or cessation of the blood supply to tissues and critical organs.

There is a need to reduce the prevalence of CVD, POAD, atherosclerosis and atheroma in human populations. There is a further need to achieve this in a cost effective manner.

GB2307178 (Broderick) discloses a magnotherapy device which includes the placement of magnets of at least three poles in a N-S-N or S-N-S configuration in the area of tissues and maintaining the position with a band or strap. The invention of GB2307178 relies on the passage of blood through an alternating magnetic field to exert a direct effect on the blood.

GB2410441 (Markland) discloses a magnotherapy device able to be magnetically clamped to the clothing of a user. This is essentially a development of GB2307178. GB2410441 discloses a N-S-N or S-N-S configuration creating CENTRAL REVERSE POLARITY™. GB2410441 relies on the passage of blood through an alternating magnetic field to exert a direct effect on the blood.

GB2343629 (Giovanni et al) discloses a system for the treatment of cellulite using pulsed magnetism. GB2343629 focuses on the design of a pair of shorts to enable positioning of a plurality of magnetic elements in either a South or North pole configuration.

U.S. Pat. No. 3,921,620 (Nakayama) discloses a band comprising a plurality of magnetic elements in either a South or North Pole configuration.

GB2325624 (Jehan) discloses a strap for trans-dermal therapy. GB2325624 focuses on a method for securing and positioning magnetic transmitters in either a South or North Pole configuration or both. Such magnetic transmitters may be either generated by fixed magnets or by electrical circuits.

US2002/0193657 (Bove) discloses a magnetic wrap for joints. The magnetic wrap has alternating polarities.

GB2367008 (Dyson and Dixon) discloses a magnetic therapy device. The inventive purpose of which relies on the passage of blood through a magnetic field to exert a direct effect on the blood and enhancing local blood flow.

GB2367007 (Lindholt) discloses a therapy device using combined and dynamic magnetic fields.

Other methods involving the positioning of magnets on the body are disclosed in JP11-350212A (Shimada), JP11-056963A (Yoshida), JP10-225497A (Kusaba), and JP9-248345A (Ishida).

U.S. Pat. No. 6,676,591 (Price) discloses a magnotherapy product comprising a magnet having positive and negative poles, the magnet being held in a housing which has attachment means for attachment to an article of clothing, and wherein a metallic element is provided adjacent the positive pole of the magnet, and a surface thereof facing the positive pole is substantially the same size as the positive pole so as to cover it, whereby the metallic element distorts the magnetic field distribution around the magnet so as to attenuate the magnetic field in the vicinity of the positive pole of the magnet.

### SUMMARY

Surprisingly, the inventors have found that application of a south-seeking magnet to a living body results in a decrease in blood inflammatory markers, in particular antigens of von Willebrand's Factor (vWF:Ag), and a decrease in blood coagulation markers in particular Platelet Aggregation Factor. These observations indicate that a specific target organ of magnotherapy is the vascular endothelium, which has been shown to be a paracrine and autocrine organ, having a central role in the control of local blood flow and a profound effect on platelet function. This effect is particularly evident with use of a magnet comprising a metallic element which distorts the magnetic field distribution around the magnet so as to attenuate the magnetic field in the vicinity of the positive pole of the magnet. The same effect was not found with magnets of other polarities.

These observations were made in a group of subjects with Restless Legs syndrome (RLS) following magnetic therapy with a magnet comprising a metallic element which distorts the magnetic field distribution around the magnet so as to attenuate the magnetic field in the vicinity of the positive pole of the magnet (this is also known as Magno-Pulse® South-seeking configuration), also known as ‘negative’.

Therefore, the application of a magnet to a living body may be used to treat inflammatory disorders, particularly those defined by an increase in vWF, blood coagulation disorders, particularly those defined by an increase in Platelet Aggregation Factor, and RLS.

According to at least one aspect of the present application relates to the use of a magnotherapy product in the treatment and/or prevention of a first or subsequent/recurrent incident of a blood coagulation disorder, wherein the magnotherapy product comprises a magnet having positive and negative poles, the magnet having a metallic element which distorts the magnetic field distribution around the magnet so as to attenuate the magnetic field in the vicinity of the positive pole of the magnet.

Optionally, according to at least one aspect of the present application, a method of treating and/or preventing of a first or subsequent/recurrent incident of a blood coagulation disorder comprising applying a magnotherapy product to a human or animal body wherein the magnotherapy product comprises a magnet having positive and negative poles, the magnet having a metallic element which distorts the magnetic field distribution around the magnet so as to attenuate the magnetic field in the vicinity of the positive pole of the magnet.

A blood coagulation disorder may be defined by an increase or decrease in Platelet Aggregation Factor compared to a reference, such as an established laboratory normal population reference range.

Optionally, a blood coagulation disorder may be linked with cardiovascular disease. The blood coagulation disorder may be selected from a bleeding disorder such as those linked with: Autoimmune disorders that produce antibodies against platelets, Bernard-Soulier syndrome, Certain medicines that block platelet formation and/or function, Glanzmann's thrombasthenia, Myeloproliferative disorders, Fibrin degradation products, Storage pool disease and Uremia (e.g. as a result of kidney failure).

In at least one embodiment blood coagulation disorders linked with cardiovascular disease show an increase in platelet aggregation factor. Bleeding disorders typically show a decrease in platelet aggregation factor.

At least one embodiment of the present application relates to the use of a magnotherapy product in the treatment and/or prevention of a first or subsequent/recurrent incident of an inflammatory condition, wherein the magnotherapy product comprises a magnet having positive and negative poles, the magnet having a metallic element which distorts the magnetic field distribution around the magnet so as to attenuate the magnetic field in the vicinity of the positive pole of the magnet.

At least one aspect of the present application also provides a method of treating and/or preventing a first or subsequent/recurrent incident of an inflammatory condition comprising applying a magnotherapy product to a human or animal subject wherein the magnotherapy product comprises a magnet having positive and negative poles, the magnet having a metallic element which distorts the magnetic field distribution around the magnet so as to attenuate the magnetic field in the vicinity of the positive pole of the magnet.

An inflammatory condition may be characterised by an increase in von Willebrand's Factor, compared to a reference, such as an established laboratory normal population reference range. The inflammatory condition may be selected from the group consisting of cardiovascular disease, rheumatological disease, inflammatory bowel disease, atherosclerosis and psoriasis. The inflammatory condition may be characterised by a decrease in von Willebrand's Factor compared to a reference, such as an established laboratory normal reference range. For example, the inflammatory condition may be von Willebrand's disease.

The ranges of magnetic strength are optimised for clinical effect while balancing the requirements of magnet size, weight and attraction of ferrous material.

Yet another aspect of the invention relates to the use of a magnotherapy product in the treatment and/or prevention of a first or subsequent/recurrent incident of damage to vascular endothelium, wherein the magnotherapy product comprises a magnet having positive and negative poles, the magnet having a metallic element which distorts the magnetic field distribution around the magnet so as to attenuate the magnetic field in the vicinity of the positive pole of the magnet.

Another aspect of the present application also provides a method of treating and/or preventing a first or subsequent/recurrent incident of damage to vascular endothelium comprising applying a magnotherapy product applying to a human or animal body wherein the magnotherapy product comprises a magnet having positive and negative poles, the magnet having a metallic element which distorts the magnetic field distribution around the magnet so as to attenuate the magnetic field in the vicinity of the positive pole of the magnet.

Damage to the vascular endothelium may be defined by an increase in von Willebrand's Factor compared to a reference, such as an established laboratory normal population reference range. For example the damage to the vascular endothelium may be indicative of coronary heart disease, peripheral vascular disease or other diseases which show atherosclerotic lesions and/or endothelial damage.

Yet another aspect of the present application relates to the use of a magnotherapy product in the treatment and/or prevention of a first or subsequent/recurrent incident of Restless Legs Syndrome, wherein the magnotherapy product comprises four magnets having positive and negative poles, each magnet having a metallic element which distorts the magnetic field distribution around the magnet so as to attenuate the magnetic field in the vicinity of the positive pole of the magnet.

At least one aspect of the present application also provides a method of treating and/or preventing a first or subsequent/recurrent incident of Restless Legs Syndrome, comprising applying a magnotherapy product to a human or animal body wherein the magnotherapy product comprises four magnets having positive and negative poles, each magnet having a metallic element which distorts the magnetic field distribution around the magnet so as to attenuate the magnetic field in the vicinity of the positive pole of the magnet.

According to at least one aspect of the present application, use of the magnotherapy product to treat and/or prevent blood coagulation disorder and/or an inflammatory condition and/or damage to the vascular endothelium and/or RLS reduces and/or eliminates the requirement for drugs and therefore may reduce the overall cost of treatment of a subject. Furthermore, there is no need to determine the level of drugs required for a given subject. The use also reduces and/or eliminates side effects associated with such drugs.

A magnotherapy of the present invention facilitates normalisation of physiological systems by promoting normal homeostasis function. That is, for a condition that is characterised by a decrease in the level of a physiological marker (e.g. platelet aggregation factor or vWF), the treatment increases the level of the physiological marker to bring it towards or to a normal level. Similarly, for a condition that is characterised by an increase in the level of a physiological marker (e.g. platelet aggregation factor of vWF), the treatment decreases the level of the physiological marker to bring it towards or to a normal level.

According to certain aspects of the present application, the magnotherapy product is preferably applied to a human or animal body, for example a body part selected from a knee, calf, abdomen, ankle and wrist.

According to at least one embodiment, the magnotherapy product is applied below a knee of a human or animal body.

According to another optional embodiment, the magnotherapy product is applied to the body for at least three hours, preferably for as long as can be tolerated by the user, more preferably 24 hours per day. This provides effective treatment of a condition. Following successful treatment, the magnotherapy product should be used for a few hours per day, e.g. when sleeping, to maintain the therapy and prevent recurrence of the condition. The magnotherapy product may be applied to the body for 24 hours per day.

Optionally, the magnotherapy product is applied to the body every day. For treatment purposes, the magnotherapy is preferably applied to the body continuously for 3 months, although it may be removed during bathing. When used for preventative purposes, the use of the magnotherapy product is preferably applied to the body for a few hours per day, e.g. overnight, and the application not interrupted for a continuous period of more than two weeks, and more preferably not interrupted for a continuous period of more than two days.

According to at least one embodiment, the magnotherapy product may be applied unilaterally to the body, i.e. to a single limb or leg. More preferably, the magnotherapy product as applied bilaterally to the body, i.e. to two limbs or legs. The term ‘limb’ includes legs and arms.

According to at least one embodiment, if a magnotherapy is applied unilaterally, i.e. applied to a single leg or limb, preferably it is used on that limb only and not transferred to the opposing leg or limb. However, if bilateral application, i.e. applied to both legs or limbs, is required, a second magnotherapy product may be applied to the second leg or limb, Transfer of unilateral therapy from a first leg or limb to the opposing leg or limb may cause adverse effects.

According to at least one embodiment, a magnotherapy product comprises a plurality of magnets. For example the magnotherapy product may comprise 2, 3, 4, 5, or 6 magnets. A magnotherapy product comprising four magnets is preferred. The use of a plurality of magnets maximises the clinical effect of the magnotherapy product.

According to at least one embodiment, the strength of magnet may be varied in accordance to the condition being treated. For example, the strength may be above 750 G, preferably from 750 G to 5,000 G, more preferably from 800 G to 3,600 G as measured at the surface of the wrap. A particularly preferred magnet has a strength of 2,000 G. It is further preferred that the magnet comprises a directional plate to enhance and focus the magnetic field towards the user. The ranges disclosed are optimised for clinical effect while balancing the requirements of magnet size, weight and attraction of ferrous material. A particularly preferred magnet has a strength of 2,000 G.

According to at least one embodiment, one or more of the magnets comprises neodymium.

### BRIEF DESCRIPTION OF THE DRAWINGS

FIG. 1 is a graph showing a significant reduction in vWF antigen in patients with RLS following one month's treatment with unilateral magnetic therapy.

FIG. 2 is a graph showing a decrease in the levels of (a) Platelet Monocyte Aggregates and (b) Platelet P-Selectin levels in patients with Restless Legs syndrome following one month's treatment with unilateral magnetic therapy.

### DETAILED DESCRIPTION

### Example 1

### Use of Wrap to Treat RLS

A wrap was prepared containing four powerful neodymium magnets (2000 G), each magnet having unique directional plates as described in U.S. Pat. No. 6,676,591. These magnets allow a negative (south-facing) enhanced magnetic field to be absorbed deeper into body tissues. It is thought that this gives a more effective and longer lasting effect in comparison to a ‘north-facing’ magnet orientation and/or the absence of the directional plate.

The wraps were fitted below the knee and above the calf muscle (not under compression) of a human subject and were held in place by “hook and loop” fastening tape. The leg wraps were double lined for comfort, and are adjustable and washable. The product is registered as a Class 1 Medical Device in the United Kingdom.

Six subjects with a clinical diagnosis of RLS were assessed prior to magnetic therapy and a comprehensive haematology coagulation profile obtained at baseline and following one month of unilateral therapy (i.e. applied to a single leg or limb) applied to the most affected leg. Blood was collected from the antecubital vein by an experienced phlebotomist to minimise coagulation effects due to the procedure. All assays were performed in a strict scientific manner and blinded from the investigators. The assays were performed according to established and accepted protocols in National Healthcare Institution and University facilities by experienced staff that were blinded to the origin and purpose of the samples.

Qualitative Results All subjects voluntarily reported an improvement of RLS in terms of sleep; both in quality and duration, pain, general wellbeing, and perceived health.

These observations are in keeping with a recent prospective observational survey completed by Dr N. Eccles of the Chiron Clinic, Harley Street, London in which 459 subjects with an existing diagnosis of RLS were enrolled in an open-label intention to treat study. No exclusions were made but they had to have had a diagnosis of RLS according to the criteria above. Subjects were asked to complete a questionnaire that requested them to report the duration of symptoms and to rate their symptoms (on a scale of 0 to 10 (where 1 represented mild symptoms and 9 to 10 more severe symptoms)); prior to and after one month of using a magnotherapy wrap. The magnotherapy wrap was equivalent to the magnotherapy product used in the present invention. Symptoms rated were: level of pain, tingling, sleep disturbance, quality of life as well as overall symptoms. They were also asked questions about their experience with other treatments, both conventional and non-conventional that they had tried previously. Patients were instructed to wear the wrap for 24 hours per day (except during bathing) for the duration of the one-month. Two wraps were supplied for bilateral application, (i.e. application to both legs or limbs).

In the Eccles observational study, it was found that, after analysing the symptoms reported before and after the use of the wrap, there was a statistically significant reduction p<0.001 for all core symptoms associated with RLS (pain, tingling, loss of sleep) with overall symptoms being reduced by 50% (p<0.001). These findings suggest enhanced efficacy over the level of relief that these subjects had previously obtained with pharmaceutical (20%, p<0.001) and other non-pharmaceutical management (10%, p<0.001). Some two-thirds of subjects had greater than 30% improvement and almost half had greater than 50% improvement in symptoms. Moreover, statistically significant improvements in quality of life were also reported.

The same group of subjects were also asked to report retrospectively by a further questionnaire after five months' use of the wrap. At this time there was evidence of further reduction of symptom severity.

The results were also measured quantitatively by measuring levels of von Willebrand's factor (vWF) and platelet aggregation factors, as shown in Example 2.

### Example 2

### Measurement of Levels of von Willebrand's Factor

vWF is a shear-induced plasma glycoprotein, which is normally synthesised by platelets, endothelial cells and megakaryocytes. vWF promotes thrombus formation by mediating adhesion of platelets to the site of injury on the vessel wall and to each other. The plasma concentration and activity of vWF are influenced by several factors, including blood group, inflammation, and proteolysis which reflect the degree of platelet activation; and hence may be used as a marker of vascular injury (Ruggeri Z M. 1997, von Willebrand's Factor, J Clin Invest 99(4): 559-64).

Several studies have shown that high vWF antigen levels are a risk factor for arterial thrombosis (Thompson S G et al. 1995, Hemostatic factors and the risk of myocardial infarction or sudden death in patients with angina pectoris. European concerted action on thrombosis and disabilities angina pectoris study group. N Engl J. Med. 1995; 332: 635-641) and for cerebrovascular disease such as stroke (Bongers et al. 2006, High von Willebrand's Factor Levels Increase the Risk of First Ischemic Stroke Influence of ADAMTS13, Inflammation, and Genetic Variability, Stroke; 37: 2672-2677).

Levels of von Willebrand's factor antigen (vWF:Ag) were measured using a commercial polyclonal rabbit antibody. Microtitre plates were coated with antibody against vWF:Ag and dilutions of standard, control and test plasmas were added. During a one-hour incubation any vWF:Ag present bound to the antibody. The plates were then washed in buffer and a second antibody directed against vWF:Ag, but coupled with an enzyme (peroxidase), was added. After a final wash, a substrate was added which is capable of generating a colour reaction. The greater the colour, the more vWF:Ag is bound to the plate and therefore the higher the plasma level. The results were interpolated from a standard curve, which was run simultaneously with the patient samples.

One suitable method of measuring vWF levels is outlined below:
a) Dilute rabbit anti-human antibody (“catch”) 1:500 in coating buffer i.e. 30 μl of antisera in 15 ml of phosphate buffer.

Phosphate Coating Buffer

| | | |
|---|---|---|
| NaH₂PO₄•2H₂O | 0.0025 M | 0.39 g |
| Na₂HPO₄•12H₂O | 0.0075 M | 2.68 g |
| NaCl | 0.145 M | 8.474 g |

b) Using a manual multihead pipette, place 100 μl of diluted catch in each well of a multiwell microtitre plate, cover with parafilm and leave overnight at 4° C.
c) Make up a 3% PEG buffer (i.e. 3.0 g PEG 8000 in 100 ml high salt buffer).
   Reconstitute Normal Reference Plasma (Technoclone, Dorking, UK) according to the manufacturer's instructions.

Wash (high salt) buffer

| | | |
|---|---|---|
| NaH₂PO₄•2H₂O | 0.0025 M | 0.78 g |
| Na₂HPO₄•12H₂O | 0.0075 M | 5.36 g |
| NaCl | 0.5 M | 58.44 g |

Make up to 2 litres with sterile dH₂O at pH 7.2. Add 4 ml of Tween 20. Store at 4° C. in fridge.
d) Make a plan of the plate.
e) Prepare the standard stock solution 1:80 by diluting 50 μl Normal Reference Plasma in 4 ml of PEG buffer. From this stock solution, prepare a standard curve in plastic test tubes:

| Concentration | | Row |
|---|---|---|
| Blank | A | PEG Buffer |
| 125% | B | Stock solution |
| 100% | C | 800 μl stock + 200 μl PEG buffer |
| 75% | D | 600 μl stock + 400 μl PEG buffer |
| 50% | E | 400 μl stock + 600 μl PEG buffer |
| 25% | F | 200 μl stock + 800 μl PEG buffer |
| 12.5% | G | 100 μl stock + 900 μl PEG buffer |
| 6.25% | H | 50 μl stock + 950 μl PEG buffer |

f) To make the dilutions manually, dilute the control and test samples as follows in labelled plastic test tubes:

| | |
|---|---|
| 1:100 | 20 μl plasma in 2 ml PEG buffer |
| 1:200 | 400 μl of 1:100 in 400 μl PEG buffer |

g) If using an auto diluter an intermediate 1-10 dilution must be used due to the syringe sizes on the autodiluter.
h) Shake out the contents of the plate and blot vigorously onto a wad of paper towels.
   Using an automated electronic 8 channel pipette (Biohit), fill each well with 200 μl of wash buffer. Leave for 3 minutes, shake out the contents and blot. Repeat this procedure twice.
i) Place 100 μl of each standard and test dilution (in duplicate) into the wells of the microtitre plate.
j) Cover the microtitre plate with parafilm and leave for 1 hour at room temperature on the bench.
k) Wash the plate three times with wash (high salt) buffer, (see step (c) for composition).
l) Dilute the “tag” antibody 1:1000 in PEG buffer i.e. 15 μl of antibody in 15 ml of PEG buffer. Using the manual multihead pipette add 100 μl of diluted “tag” to each well of the microtitre plate. Cover with parafilm and leave for 1 hour at room temperature.
m) Wash the microtitre plate three times.
n) Make up the colour reagent solution in a 25 ml plastic container:
   15 ml Substrate buffer
   10 mg Orthophenylenediamine tablet (OPD).
o) Exclude light from the container by wrapping a paper towel secured with a rubber band around it. Allow 10 minutes for the OPD tablet to dissolve.
p) Just before use add 7 μl 30% H₂O₂.
q) Add 100 μl of colour reagent to each well using the manual multihead pipette. Leave the plate uncovered on the bench for 7 mins.
r) NOTE: the strength of the antisera varies from batch to batch. The colour may take less time to develop, so look at the plate occasionally. Once a difference in colour can be seen in the lowest dilutions of the standard curve, the reaction may be stopped.
s) Stop the reaction by adding 100 μl of 1.5 M sulphuric acid to the wells. Add the sulphuric acid in the same order as the colour reagent was added to prevent discrepancies.
t) Leave the plate for ten minutes at room temperature to allow any bubbles to disperse. Read the absorbencies at 492 nm on a KC4 microtitre plate reader.

Using the above method, a standard curve may be drawn using KC4 plate reader software. The same software may be used to automatically calculate values for each well. The average total vWF concentration may be calculated from the Coagulation Reference from all four dilutions. If the Coagulation Reference value is within 10% of the target value, average the patient's results and report.

The Coagulation Reference is a known standard used for internal quality control.

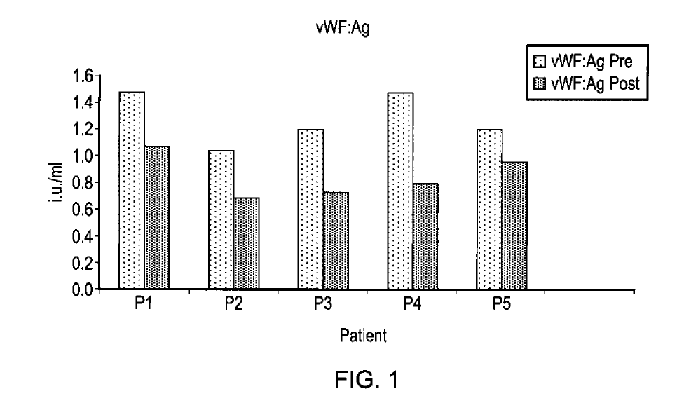

Patients with RLS were treated with unilateral magnetic therapy for one month. vWF antigen levels were measured prior to and following the treatment. As shown in FIG. 1., there was a significant reduction in vWF antigen following one month's treatment.

The horizontal dashed lines represent the upper and lower normal ranges of vWF. Of the five patients reported below, all demonstrated a significant decrease in vWF:Ag following therapy. Importantly, four had abnormally elevated vWF at baseline, which decreased to normal following therapy.

### Example 3

### Measurement of Platelet Aggregation

Platelet Aggregation Long recognized as having a role in inflammation, platelets and platelet-leukocyte aggregates are now known to contribute to ongoing injury at atheromatous sites, and in plaque disruption. Platelet P-Selectin (CD62P) interacts with its natural ligand on neutrophils and monocytes, and PSGL-1 receptor, to allow formation of platelet neutrophil and platelet monocyte aggregates, thus providing an anchoring source for inflammatory cells on activated platelets.

These bioactive platelet-monocyte aggregates have been shown to be important in cardiovascular disease in humans as they are involved in ongoing vascular inflammation and thrombosis.

The measurement of indicators of platelet activation during routine haematological investigations offers advantages in the clinical evaluation and management of patients at risk from thrombotic and other diseases. The assays were performed in accordance to the protocols designed and reported in Macey et al 2003, Ethylenediaminetetraacetic acid plus citrate-theophylline-adenosine-dipyridamole (EDTA-CTAD): a novel anticoagulant for the flow cytometric assessment of platelet and neutrophil activation ex vivo in whole blood. Cytometry B Clin Cytom.: 51(1): 30-40. The method is summarised below:

Materials: K₃EDTA and CTAD in Vacutainers® were from BD Biosciences (Cowley, Oxford, UK); the latter were stored in light protective boxes and removed just prior to use.

Blood samples: Blood was collected from the antecubital vein.

Assessment of platelet activation: Whole blood samples were taken into Vacutainers® that contained a mixture of K₃EDTA and CTAD. Blood was collected first into K₃EDTA and then immediately transferred to a Vacutainer® containing CTAD. Samples were held at ambient temperature, analysed within two hours of venesection. The platelet count (PLT), mean platelet volume (MPV) and mean platelet component concentration (MPC) was determined using the ADVIA®120 haematology system (Bayer Corporation, Tarrytown, N.Y.). Platelet counts were corrected for dilution. The system was calibrated and standardised prior to use with ADVIA®-SETpoint Haematology Control and ADVIA® OPTIpoint, respectively (Bayer Corporation).

Measurement of the percentage platelets expressing CD62P: Anticoagulated blood (5 μl) was labelled at ambient temperature with FITC-isotype control (5 μl), FITC-CD62P (5 μl), PE-CD45 (5 μl) and FITC-isotype control (5 μl), or PE-CD45 (5 μl) and FITC-CD42a (5 μl), in 90 μl of TS for 5 min.

Flow cytometry: Blood cells were analysed on a FACScan (BD Biosciences) equipped with CellQuest® software. The flow cytometer was calibrated and standardised prior to use with fluorochrome labelled beads (Fluorospheres; Dako, Ely, Cambridgeshire, UK).

For the analysis of CD62P expression, data was acquired in real time with a primary gate set on a dual parameter histogram of forward light scatter logarithmic scale (abscissa) and side light scatter logarithmic scale (ordinate). Background fluorescence was assessed with platelets labelled with the FITC-conjugated isotype control antibody. Cursors were set in a single parameter dot plot of frequency (ordinate) and green fluorescence intensity (abscissa), so that less than 1% of the platelets stained positively with the control antibody. Changes in CD62P expression (green fluorescence logarithmic scale), together with those of forward and side light scatter, and were then recorded on the gated platelets.

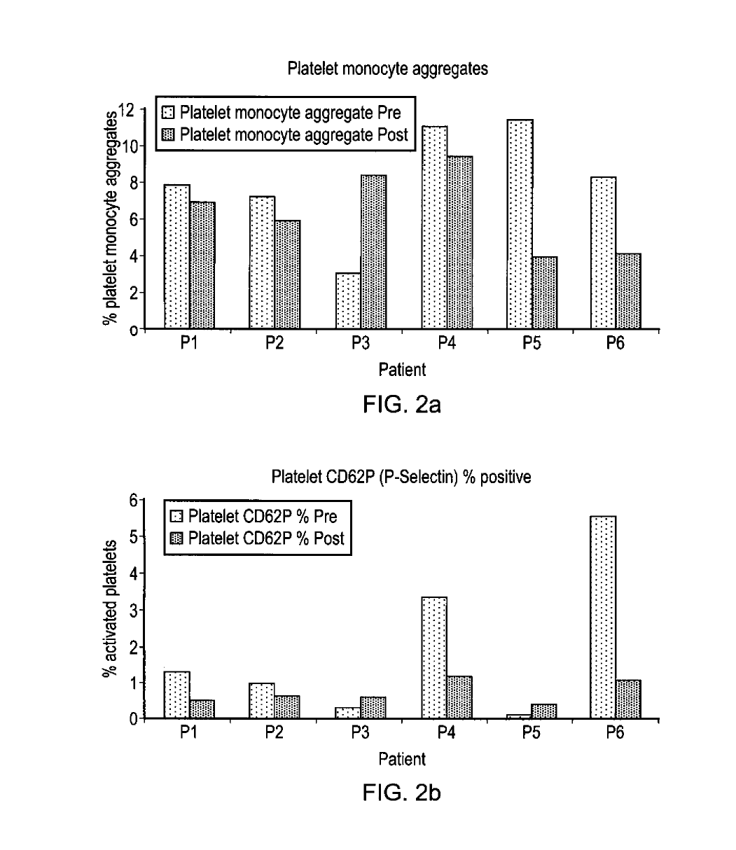

FIG. 2. shows alterations in the levels of (a) Platelet Monocyte Aggregates and (b) Platelet P-Selectin levels in patients with Restless Legs Syndrome. The horizontal dashed line represents the upper normal ranges.

FIG. 2a shows that five of the subjects demonstrated a fall in platelet monocyte aggregates. Importantly two had abnormally elevated monocyte aggregates at baseline, which decreased to normal following therapy.

FIG. 2b shows that four of the subjects demonstrated a fall in aggregates; two had abnormally elevated platelet P-Selectin expression at baseline, which decreased to normal following therapy.

Thus a method and use of magnotherapy has been described. It will be appreciated that the method has been described in terms of several embodiments, which are susceptible to various modifications and alternative forms. Accordingly, although specific embodiments have been described, these are examples only and are not limiting upon the scope of the invention.

1. A method of treating and/or preventing a blood coagulation disorder comprising applying a magnotherapy product to a human or animal body in which the magnotherapy product comprises a magnet having positive and negative poles, the magnet having a metallic element which distorts the magnetic field distribution around the magnet so as to attenuate the magnetic field in the vicinity of the positive pole of the magnet.

2. A method according to claim 1 in which the blood coagulation disorder is defined by an increase in Platelet Aggregation Factor, compared to an established laboratory normal population reference range.

3. A method according to claim 1 or 2 in which the blood coagulation disorder is linked with cardiovascular disease.

4. A method according to claim 1 in which the blood coagulation disorder is defined by a decrease in Platelet Aggregation Factor, compared to an established laboratory normal population reference range.

5. A method according to claim 1 or 4 in which the blood coagulation disorder is a bleeding disorder.

6. A method according to claim 5 in which the bleeding disorder is selected from those linked with: Autoimmune disorders that produce antibodies against platelets, Bernard-Soulier syndrome, use of certain medicines that block platelet formation and/or function, Glanzmann's thrombasthenia, Myeloproliferative disorders, Fibrin degradation products, Storage pool disease and Uremia.

7. A method of treating and/or preventing an inflammatory condition comprising applying a magnotherapy product to a human or animal body in which the magnotherapy product comprises a magnet having positive and negative poles, the magnet having a metallic element which distorts the magnetic field distribution around the magnet so as to attenuate the magnetic field in the vicinity of the positive pole of the magnet.

8. A method according to claim 7 in which the inflammatory condition is characterised by an increase in von Willebrand's Factor, compared to an established laboratory normal population reference range.

9. A method according to claim 7 or 8 in which the inflammatory condition is selected from the group consisting of cardiovascular disease, a rheumatological disease, inflammatory bowel disease, atherosclerosis and psoriasis.

10. A method according to claim 7 in which the inflammatory condition is characterised by a decrease in von Willebrand's Factor, compared to an established laboratory normal population reference range.

11. A method according to claim 7 or 10 in which the inflammatory condition is von Willebrand's disease.

12. A method of treating and/or preventing damage to vascular endothelium comprising applying a magnotherapy product to a human or animal body in which the magnotherapy product comprises a magnet having positive and negative poles, the magnet having a metallic element which distorts the magnetic field distribution around the magnet so as to attenuate the magnetic field in the vicinity of the positive pole of the magnet.

13. A method according to claim 12 in which the damage to the vascular endothelium is characterised by an increase in von Willebrand's Factor.

14. A method according to claim 12 or 13 in which damage to the vascular endothelium is indicative of coronary heart disease, peripheral vascular disease or another disease which shows a characteristic selected from the group consisting of atherosclerotic lesions and endothelial damage.

15. A method of treating and/or preventing Restless Legs Syndrome, comprising applying a magnotherapy product to a human or animal body in which the magnotherapy product comprises four magnets having positive and negative poles, each magnet having a metallic element which distorts the magnetic field distribution around the magnet so as to attenuate the magnetic field in the vicinity of the positive pole of the magnet.

16. A method according to any one of claims 1 to 15 in which the magnotherapy product is applied to one or more regions of the body selected from the group consisting of: knee, calf, abdomen, ankle and wrist.

17. A method according to any one of claims 1 to 15 in which the magnotherapy product is applied below a knee of the body.

18. A method according to any one of claims 1 to 17 in which the magnotherapy product is applied to the body for at least three hours per day.

19. A method according to claim 18 in which the magnotherapy product is applied to the body for twenty four hours per day.

20. A method according to any one of claims 1 to 14 and 16 to 19 in which the magnotherapy product comprises a plurality of magnets.

21. A method according to claim 20 in which the magnotherapy product comprises four magnets.

22. A method according to any one of claims 1 to 21 in which one or more of the magnets has a strength of 2,000 G.

23. A method according to any one of claims 1 to 22 in which one or more of the magnets comprises neodymium.

24. A method according to any one of claims 1 to 24 in which the body is a human.
